(19) United States
(12) Patent Application Publication
(10) Pub. No.: US 2024/0262770 A1
(43) Pub. Date: Aug. 8, 2024

(54) PROCESS FOR PRODUCING METHANOL FROM SYNTHESIS GAS COMPRISING A HIGH PROPORTION OF INERT GAS COMPONENTS

(71) Applicant: L'Air Liquide, Société Anonyme pour l'Etude et l'Exploitation des Procédés Georges Claude, Paris (FR)

(72) Inventors: Stéphane HAAG, Frankfurt am Main (DE); Thomas RENNER, Frankfurt am Main (DE); Frank CASTILLO-WELTER, Friedrichsdorf (DE); Nga Thi Quynh DO, Frankfurt am Main (DE)

(21) Appl. No.: 18/435,360

(22) Filed: Feb. 7, 2024

(30) Foreign Application Priority Data
Feb. 7, 2023 (EP) 23155309.0

(51) Int. Cl. C07C 29/151; C07C 29/76

(52) U.S. Cl. CPC C07C 29/1512; C07C 29/76

(57) ABSTRACT

The invention relates to a process for producing methanol in which at least a portion of the obtained liquid raw methanol stream is decompressed to a decompression pressure in a liquid decompression apparatus, wherein the decompression pressure is lower than the synthesis pressure and wherein the liquid decompression apparatus does mechanical work. At least a portion of the mechanical work done by the liquid decompression apparatus is utilized to drive the synthesis gas compression apparatus required for compressing the synthesis gas to synthesis pressure. The utilization may be carried out indirectly, through generation of electrical energy, or directly through direct utilization of the mechanical work.

### CROSS REFERENCE TO RELATED APPLICATIONS

This application claims the benefit of priority under 35 U.S.C. § 119 (a) and (b) to European Patent Application No. 23155409.0, filed Feb. 7, 2023, the entire contents of which are incorporated herein by reference.

### BACKGROUND

### Technical Field of the Invention

The invention relates to a process for producing methanol from synthesis gas, in particular from synthesis gas comprising a high proportion of inert gas components.

### Prior Art

Methanol is produced on an industrial scale from synthesis gas, a mixture of carbon oxides and hydrogen. Synthesis gas is typically produced from fossil input gases, such as natural gas, through processes such as steam reforming (SMR) or autothermal reforming (ATR). These are established industrial processes which produce a synthesis gas mixture that consists almost exclusively of carbon monoxide, carbon dioxide and hydrogen. Inert constituents such as methane or nitrogen make up only a small proportion of the synthesis gas mixture.

Methanol may be produced on the basis of carbon dioxide-rich input gases using high recycle rates or in multi-stage syntheses with intermediate condensation of the raw methanol also from practically carbon monoxide-free input gases. In the latter case the actual synthesis gas converted into methanol is thus a mixture of carbon dioxide and hydrogen.

To reduce industrially generated carbon dioxide emissions carbon dioxide may either be separated and sequestered (carbon capture and storage, CCS) or after separation be sent to a further utilization (carbon capture and utilization, CCU). CCU lends itself to methanol synthesis based on carbon dioxide-rich input gases, for example based on industrial offgases. Such input gases may at least be blended with “conventional” synthesis gas since as such, even after addition of hydrogen, they are not always directly suitable for methanol synthesis.

However, the aforementioned input gases generally comprise a high proportion of inert constituents, more precisely a high proportion of gas components that are inert, i.e. do not react, under methanol synthesis conditions and thus do not contribute to product formation. This presents a problem when using high recycle rates in conventional single-stage methanol syntheses, since inert components accumulate at the reactor inlet, thus considerably increasing the reactor load, causing a high pressure drop across the reactor and reducing the yield based on carbon conversion. This also increases the load on the synthesis gas compressor and the recycle gas compressor, thus resulting in higher operating costs (opex). The cross sections of the pipes must also be made correspondingly large, thus increasing capital costs (capex).

The abovementioned problems in some cases also apply to multi-stage methanol syntheses with intermediate condensation of the raw methanol. These are typically configured such that virtually the entirety of the carbon of the synthesis gas is converted into methanol in a single pass through all reactor stages. A recycling of the unconverted synthesis gas is thus not necessary in many cases.

In order to be able to utilize input gases with high proportions of inert constituents for a methanol synthesis it is generally necessary for the aforementioned reasons to undertake a purification of the input gas, i.e. a costly and complex separation of the inert constituents, before making it available to the synthesis.

### SUMMARY

In many cases it must be assumed that a costly and complex separation of inert gas constituents from the respective starting material is not worthwhile for economic reasons. It is therefore necessary to find another way to operate a corresponding methanol production in a technically and economically sensible fashion even at a high proportion of inert constituents in the input gas.

The synthesis gas compressor, i.e. the compressor which compresses the synthesis gas mixture to the synthesis pressure required for methanol synthesis, plays a central role. This is generally a machine operated with a high input of electrical energy.

The objective should be to keep constant or reduce the amount of externally generated electrical energy for the synthesis gas compressor that is to be provided even in operation with an input gas comprising a high proportion of inert constituents relative to operation of the synthesis gas compressor with an input gas without a high proportion of inert constituents. Such a result is achievable for example through improved process integration when for example energy generated process-internally is utilized in such a way that it reduces the outlay for providing energy generated process-externally.

EP 2 228 357 A1 discloses a process for producing methanol in which a purge gas stream is decompressed using an expander. The mechanical work generated is utilized process-internally, for example to drive a recycle gas compressor.

The disadvantage of this is that the purge gas stream within a methanol synthesis with recycling of the unconverted synthesis gas accounts for only a relatively small partial volume flow relative to the total volume flow of the residual gas unconverted in the reactor and the recovery of energy is correspondingly limited to this partial volume flow.

However, when using input gases having a high proportion of inert gas components it would be desirable to be able to utilize the greatest possible proportion of the gas stream for process-internal energy recovery or to generate additional possibilities for process-internal energy recovery. It is in any case an objective to minimize the energy input for the synthesis gas compressor.

It is accordingly an object of the present invention to at least partially overcome the aforementioned disadvantages of the prior art.

It is especially an object of the present invention to expand or improve the possibilities for process-internal utilization of energy.

It is a further object of the present invention to further improve the process integration of methanol production having regard to external energy demand so that methanol production may be realized in a technically and economically sensible fashion even with input gases comprising a high proportion of inert constituents without the need to effect a preceding separation of the inert constituents from the respective input gas.

The independent claims make a contribution to the at least partial achievement of at least one of the above objects. The dependent claims provide preferred embodiments which contribute to the at least partial achievement of at least one of the objects. Preferred embodiments of constituents of one category according to the invention are, where relevant, likewise preferred for identically named or corresponding constituents of a respective other category according to the invention.

The terms “having”, “comprising” or “containing” etc. do not preclude the possible presence of further elements, ingredients etc. The indefinite article “a” does not preclude the possible presence of a plurality.

A first aspect of the invention proposes a process for producing methanol, comprising the process steps of
a) providing a synthesis gas stream comprising at least one carbon oxide, hydrogen and an inert gas component;
b) compressing the synthesis gas stream to synthesis pressure in a synthesis gas compression apparatus;
c) reacting the compressed synthesis gas stream over a methanol synthesis catalyst to afford a product stream, wherein the product stream comprises at least methanol, water, unconverted synthesis gas and the inert gas component, wherein the reacting of the compressed synthesis gas stream is carried out in a reactor arrangement, wherein the reactor arrangement comprises at least one reactor stage and a phase separation apparatus;
d) separating the product stream in the phase separation apparatus into a liquid raw methanol stream, comprising at least methanol and water, and a residual gas stream comprising at least unreacted synthesis gas and the inert gas component;
e) decompressing at least a portion of the raw methanol stream in a liquid decompression apparatus to a decompression pressure which is lower than the synthesis pressure, wherein the liquid decompression apparatus does mechanical work;
f) transmitting at least a portion of the mechanical work done by the liquid decompression apparatus to the synthesis gas compression apparatus to drive the synthesis gas compression apparatus or
converting at least a portion of the mechanical work done by the liquid decompression apparatus into electrical energy and utilizing the electrical energy to drive the synthesis gas compression apparatus.

A reactor stage of the reactor arrangement comprises the methanol synthesis catalyst. This may be any catalyst known to those skilled in the art and suitable for methanol synthesis, for example a copper-based catalyst. The catalyst may be configured as a fixed bed in the form of pellets or as a structured packing for example.

A phase separation apparatus is configured for separating the product stream from the residual gas stream. In one example the phase separation apparatus comprises a cooling stage in the form of a heat exchanger for condensing the product stream and a separation stage for separating the condensed (liquid) phase from the gas phase.

According to the invention at least a portion of the raw methanol stream is decompressed to a decompression pressure in a liquid decompression apparatus and the mechanical work done by the decompression apparatus is utilized process-internally. The utilization may be effected directly, by mechanically driving the synthesis gas compression apparatus, or indirectly by initially converting the mechanical work done by the decompression apparatus into electrical energy. The latter is then utilized to drive the synthesis gas compression apparatus.

The residual gas stream may optionally be recompressed to synthesis pressure in a residual gas compression apparatus and combined with the compressed residual gas stream. In this optional embodiment the residual gas stream is thus recycled back to the inlet of the reactor stage and converted into the raw methanol stream with the compressed synthesis gas. The mechanical work done by the liquid decompression apparatus may optionally also be utilized at the residual gas compression apparatus to drive the residual gas compression apparatus or the mechanical work done by the liquid decompression apparatus is converted into electrical energy and the electrical energy utilized to drive the residual gas compression apparatus.

The liquid decompression apparatus is for example a decompression machine operating by the displacement principle (for example piston displacement machine) or the flow principle (for example turbo decompression machine, decompression turbine). Selection of the liquid decompression apparatus proceeds according to the volume flow of the raw methanol stream to be decompressed and the prevailing pressures.

The decompression pressure is preferably lower than the synthesis pressure by a factor of at least 5 or of at least 10 or of at least 15 or of at least 20 or of at least 25 or of at least 50. In one example the synthesis pressure is in a range from 60 bar to 90 bar and the decompression pressure is in a range from atmospheric pressure (about 1 bar absolute) to 5 bar.

If the work done by the liquid decompression apparatus is initially converted into electrical energy this may be carried out for example by a generator. The thus generated electrical current may subsequently drive, for example, an electric motor which drives the synthesis gas compression apparatus.

The synthesis gas compression apparatus is one or more parallel and/or serially arranged compressors which in turn operate either according to the displacement principle (for example piston compressor) or according to the flow principle (for example turbo compressor).

The synthesis gas stream comprises at least one carbon oxide, hydrogen and an inert gas component. A “carbon oxide” is to be understood as meaning carbon monoxide (CO) or carbon dioxide (CO₂). In terms of substances the inert gas components may be a single gas (for example exclusively nitrogen) or a mixture of gases (for example nitrogen, argon and methane). “Inert” is to be understood as meaning that the gas component does not react under the conditions of methanol synthesis, i.e. is converted neither to the product nor to an undesired byproduct.

In one embodiment, in particular in a methanol synthesis comprising only one or comprising two reactor stages, at least a portion of the residual gas stream is recycled to the inlet of the one or one of the reactor stage(s). A purge stream may be diverted from a portion of this recycle stream to prevent accumulation of the inert constituents in the synthesis loop. The purge stream may additionally be utilized for doing mechanical work and thus for process-internal energy generation with the aid of a gas decompression apparatus, such as is known from EP 2 228 357 A1.

One embodiment of the process is characterized in that the reactor arrangement comprises a plurality n of serially arranged reaction stages and a plurality p of phase separation apparatuses, wherein each of the reactor stages has a phase separation apparatus assigned to it, wherein the phase separation apparatus is in each case arranged downstream of the assigned reaction stage and in each of the phase separation apparatuses a liquid raw methanol substream and a residual gas substream are generated and wherein the residual gas substream produced in a phase separation apparatus is at least partially introduced into the respective subsequent reactor stage and wherein the residual gas substream produced in the last phase separation apparatus is discharged from the reactor arrangement.

In this embodiment the reactor arrangement comprises at least two reactor stages and at least two phase separation apparatuses. Each reactor stage has a phase separation apparatus assigned to it which is arranged downstream of the respective reactor stage. The product substream discharged from a reactor stage is separated into a raw methanol substream and a residual gas substream in the downstream phase separation stage. The raw methanol substream is discharged from the reactor arrangement as a raw product substream and subjected to a further workup. The residual gas substream withdrawn from a phase separation stage is preferably entirely introduced into the respective subsequent reactor stage of the serially arranged reactor stages. It is also possible to introduce only a portion into the respective subsequent reactor stage, the remaining portion being subjected to a further use.

The last of the serially arranged phase separation stages is an exception. The residual gas substream discharged from this last phase separation stage is discharged from the reactor arrangement and subjected to a further use.

The configuration of the reactor stages and the phase separation apparatuses within the reactor arrangement in this embodiment may also be referred to as a multi-stage reactor arrangement with intermediate condensation.

In this regard one embodiment of the process is characterized in that the process comprises the further process steps of
g) decompressing at least a portion of the residual gas substream discharged from the reactor arrangement in a gas decompression apparatus to a decompression pressure lower than the synthesis pressure, wherein the gas decompression apparatus does mechanical work;
h) transmitting at least a portion of the mechanical work done by the gas decompression apparatus to the synthesis gas compression apparatus to drive the synthesis gas compression apparatus or
converting at least a portion of the mechanical work done by the gas decompression apparatus into electrical energy and utilizing the electrical energy to drive the synthesis gas compression apparatus.

This embodiment relates to the configuration of the reactor arrangement as a multi-stage reactor arrangement. In this embodiment the residual gas substream discharged from the reactor arrangement is advantageously also used to do mechanical work when this residual gas substream is decompressed to a decompression pressure in a gas decompression apparatus

When using a number of reactor stages n that ensures that a complete or virtually complete carbon conversion is achieved over all reactor stages the residual gas substream discharged from the last phase separation stage practically no longer contains any valuable gases but rather consists substantially of the gases of the inert gas component. It is therefore especially sensible to utilize the residual gas substream for process-internal recovery of mechanical or electrical energy for driving the synthesis gas compressor. This is especially sensible in connection with a synthesis gas stream which comprises an inert gas component in a proportion of at least 1% by volume in the synthesis gas.

In contrast to the process known from EP 2 228 357 A1 this embodiment does not comprise diverting a proportion from the residual gas stream as a purge stream but rather the entire residual gas stream may be utilized for process-internal recovery of mechanical or electrical energy.

The decompression pressure of the decompressed residual gas substream is preferably lower than the synthesis pressure by a factor of at least 5 or of at least 10 or of at least 15 or of at least 20 or of at least 25 or of at least 50. In one example the synthesis pressure is in a range from 60 bar to 90 bar and the decompression pressure is in a range from atmospheric pressure (about 1 bar absolute) to 5 bar.

The gas decompression apparatus is for example a decompression machine operating by the displacement principle (for example piston displacement machine) or the flow principle (for example turbo decompression machine, decompression turbine). Selection of the gas decompression apparatus proceeds according to the volume flow of the raw methanol stream to be decompressed and the prevailing pressures.

Depending on the composition of the residual gas substream it may be sensible to supply the decompressed residual gas substream to a hydrogen recovery apparatus and to supply the hydrogen thus recovered to the methanol synthesis.

One embodiment of the process is characterized in that the entire residual gas substream discharged from the reactor arrangement is decompressed in the gas decompression apparatus.

The utilization of the entire residual gas substream discharged from the reactor arrangement for process-internal recovery of mechanical or electrical energy is sensible especially when the number n of reactor stages is high enough to ensure that over all reactor stages n of the reactor arrangement complete or substantially complete carbon conversion is achieved.

It is alternatively also possible for a proportion of the residual gas substream discharged from the reactor arrangement to be decompressed in the gas decompression apparatus and a further proportion of the residual gas substream discharged from the reactor arrangement to be compressed to synthesis pressure in a residual gas compression apparatus and the compressed residual gas substream to be recycled at least to one of the plurality of serially arranged reactor stages, preferably to the first of the plurality of serially arranged reactor stages.

This is sensible especially when the residual gas substream discharged from the last phase separation apparatus still contains a utilizable proportion of carbon oxides and hydrogen. In this case only a portion of the residual gas substream is utilized for process-internal recovery of mechanical or electrical energy while the other portion is recompressed to synthesis pressure and preferably recycled to the first of the plurality of serially arranged reactor stages.

The mechanical or electrical energy recovered process-internally through decompression of the residual gas substream may optionally also be utilized to drive the residual gas compression apparatus.

In one embodiment a portion of the residual gas substream decompressed to decompression pressure is recompressed to synthesis pressure in the synthesis gas compression apparatus and the compressed residual gas substream is supplied to the reactor arrangement.

At appropriate composition of the residual gas substream it is sensible to recycle said substream to the synthesis gas compression apparatus after decompression. Here, the residual gas substream is compressed to synthesis pressure together with the synthesis gas stream and subsequently supplied to the reactor arrangement.

One embodiment of the process is characterized in that the proportion of the inert gas component in the synthesis gas stream is at least 1% by volume or at least 5% by volume or at least 10% by volume or at least 20% by volume.

The proportion of the inert gas component in the synthesis gas stream is in particular 0.5% by volume to 30% by volume, or 1% by volume to 20% by volume, or 1% by volume to 10% by volume.

One embodiment of the process is characterized in that
the inert gas component comprises nitrogen and/or methane.

One embodiment of the process is characterized in that
the synthesis pressure is at least 70 bar, or is at least 75 bar, or is at least 80 bar, or is at least 85 bar.

One embodiment of the process is characterized in that
the raw methanol stream is decompressed to a pressure of 1 bar to 5 bar, preferably to a pressure of 1 bar to 3 bar, in the liquid decompression apparatus. In a preferred embodiment the raw methanol stream in the liquid decompression apparatus is decompressed to a pressure corresponding to atmospheric pressure, i.e. about 1 bar (absolute).

A particularly preferred embodiment of the process is characterized in that the raw methanol stream is supplied to a downstream thermal separation apparatus for separating the raw methanol into methanol and water, wherein the thermal separation apparatus is operated at a predetermined pressure and the raw methanol stream is decompressed in the liquid decompression apparatus to a pressure corresponding to the predetermined pressure in the thermal separation apparatus.

The raw methanol stream in the liquid decompression apparatus is especially decompressed to a pressure which substantially corresponds to the predetermined pressure in the thermal separation apparatus.

As a result of this measure the decompressed raw methanol stream may subsequently be directly introduced into the downstream thermal separation apparatus.

In one embodiment the thermal separation apparatus is a low-pressure phase separation apparatus. In this configuration the phase separation apparatus arranged immediately downstream of the reactor stage is configured as a high-pressure phase separation apparatus or the phase separation apparatuses arranged downstream of the reactor stages are configured as high-pressure phase separation apparatuses. The liquid decompression apparatus is then arranged between the high-pressure phase separation apparatus and the low-pressure phase separation apparatus or between the high-pressure phase separation apparatuses and the low-pressure phase separation apparatus.

In one example the thermal separation apparatus is a rectification column or an arrangement of two or more rectification columns.

One embodiment of the process is characterized in that
the residual gas substream is decompressed to a pressure of 1 bar to 5 bar, preferably to a pressure of 1 bar to 3 bar, in the liquid decompression apparatus. In a preferred embodiment the residual gas substream in the liquid decompression apparatus is decompressed to a pressure corresponding to atmospheric pressure, i.e. about 1 bar (absolute).

One embodiment of the process is characterized in that for the plurality n of serially arranged reactor stages and the plurality p of phase separation apparatuses
n=3 to 6, preferably n=4, and
p=3 to 6, preferably p=4, and wherein
n=p.

A second aspect of the invention proposes a process for producing methanol, comprising the process steps of
a) providing a synthesis gas stream comprising at least one carbon oxide, hydrogen and an inert gas component;
b) compressing the synthesis gas stream to synthesis pressure in a synthesis gas compression apparatus;
c) reacting the compressed synthesis gas stream over a methanol synthesis catalyst to afford a product stream, wherein the product stream comprises at least methanol, water, unconverted synthesis gas and the inert gas component, wherein the reacting of the compressed synthesis gas stream is carried out in a reactor arrangement, wherein the reactor arrangement comprises at least one reactor stage and a phase separation apparatus;
d) separating the product stream in the phase separation apparatus into a liquid raw methanol stream, comprising at least methanol and water, and a residual gas stream comprising at least unreacted synthesis gas and the inert gas component;
wherein the reactor arrangement comprises a plurality n of serially arranged reaction stages and a plurality p of phase separation apparatuses, wherein each of the reactor stages has a phase separation apparatus assigned to it, wherein the phase separation apparatus is in each case arranged downstream of the assigned reaction stage and in each of the phase separation apparatuses a liquid raw methanol substream and a residual gas substream are generated and wherein the residual gas substream produced in a phase separation apparatus is at least partially introduced into the respective subsequent reactor stage and wherein the residual gas substream produced in the last phase separation apparatus is discharged from the reactor arrangement; and
further comprising the process steps of
e) decompressing at least a portion of the residual gas substream discharged from the reactor arrangement in a gas decompression apparatus to a decompression pressure lower than the synthesis pressure, wherein the gas decompression apparatus does mechanical work;
f) transmitting at least a portion of the mechanical work done by the gas decompression apparatus to the synthesis gas compression apparatus to drive the synthesis gas compression apparatus or
converting at least a portion of the mechanical work done by the gas decompression apparatus into electrical energy and utilizing the electrical energy to drive the synthesis gas compression apparatus.

In the second aspect of the process the residual gas substream discharged from the reactor arrangement is advantageously also used to do mechanical work when this residual gas substream is decompressed to a decompression pressure in a gas decompression apparatus

When using a number of reactor stages n that ensures that a complete or virtually complete carbon conversion is achieved over all reactor stages the residual gas substream discharged from the last phase separation stage practically no longer contains any valuable gases but rather consists substantially of the gases of the inert gas component. It is therefore especially sensible to utilize the residual gas substream for process-internal recovery of mechanical or electrical energy for driving the synthesis gas compressor. This is especially sensible in connection with a synthesis gas stream which comprises an inert gas component in a proportion of at least 1% by volume in the synthesis gas.

In contrast to the process known from EP 2 228 357 A1 this embodiment does not comprise diverting a proportion from the residual gas stream as a purge stream but rather the entire residual gas stream may be utilized for process-internal recovery of mechanical or electrical energy.

In one embodiment of the second aspect of the process the entire residual gas substream discharged from the reactor arrangement is decompressed in the gas decompression apparatus.

In one embodiment of the second aspect of the process a proportion of the residual gas substream discharged from the reactor arrangement is decompressed in the gas decompression apparatus and a further proportion of the residual gas substream discharged from the reactor arrangement is compressed to synthesis pressure in a residual gas compression apparatus and the compressed residual gas substream is recycled at least to one of the plurality of serially arranged reactor stages, preferably to the first of the plurality of serially arranged reactor stages.

In one embodiment of the second aspect of the process a portion of the residual gas substream decompressed to decompression pressure is recompressed to synthesis pressure in the synthesis gas compression apparatus and the compressed residual gas substream is supplied to the reactor arrangement.

In one embodiment of the second aspect of the process the proportion of the inert gas component in the synthesis gas stream is at least 1% by volume or at least 5% by volume or at least 10% by volume or at least 20% by volume.

In one embodiment of the second aspect of the process the inert gas component comprises nitrogen and/or methane.

In one embodiment of the second aspect of the process the synthesis pressure is at least 70 bar or at least 75 bar or at least 80 bar or at least 85 bar.

In one embodiment of the second aspect of the process the residual gas substream is decompressed in the gas decompression apparatus to a pressure of 1 bar to 3 bar.

In one embodiment of the second aspect of the process for the plurality n of serially arranged reactor stages and the plurality p of phase separation apparatuses
n=3 to 6, preferably n=4, and
p=3 to 6, preferably p=4, and
n=p.

### BRIEF DESCRIPTION OF THE DRAWINGS

The invention is more particularly elucidated hereinbelow by exemplary embodiments. In the following detailed description reference is made to the accompanying drawings which form a part of the exemplary embodiments and which contain illustrative specific representations of specific embodiments of the invention.

In the description that follows and in the drawings, identical elements are identified by identical reference numerals. Gas streams are represented in the figures by dashed lines while liquid streams are represented by solid lines. The flow direction of the respective streams is indicated by arrows.

In the figures:

FIG. 1 shows a process 1 according to a first example of the invention comprising a single-stage methanol synthesis,

FIG. 2 shows a process 2 according to a second example of the invention comprising a four-stage methanol synthesis,

FIG. 3 shows a process 3 according to a third example of the invention comprising a four-stage methanol synthesis, and

FIG. 4 shows a process 4 according to a fourth example of the invention with a four-stage methanol synthesis

### DETAILED DESCRIPTION OF PREFERRED EMBODIMENTS

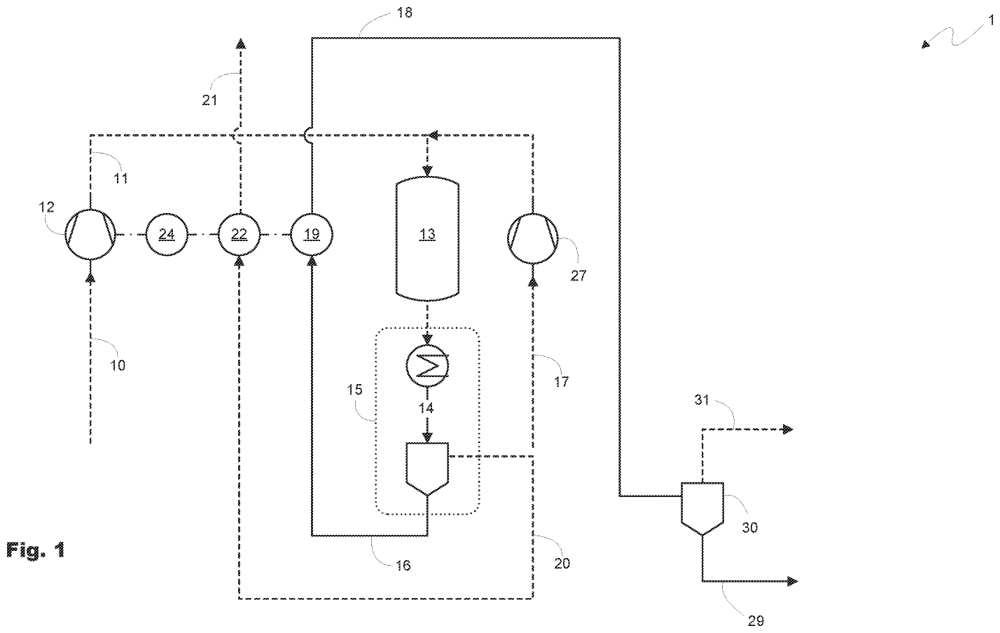

FIG. 1 shows a process 1 according to a first example of the invention in which the synthesis of the methanol is carried out in a single-stage reactor arrangement.

A synthesis gas stream 10 comprising hydrogen, carbon monoxide, carbon dioxide and also nitrogen and methane as inert gas component is provided and compressed to a synthesis pressure suitable for methanol synthesis in a synthesis gas compression apparatus 12, here a synthesis gas compressor. In one example the synthesis pressure may be 90 bar. A resulting compressed synthesis gas stream 11 is introduced into a reactor stage 13 in which the compressed synthesis gas stream 11 is converted into a product stream 14 which is gaseous initially and biphasic after cooling which contains at least methanol, water and unconverted synthesis gas (hydrogen, carbon monoxide and carbon dioxide) and the inert gas component. The reactor stage 13 contains a suitable copper-based methanol synthesis catalyst (not shown). Since the reaction of methanol formation from synthesis gas is exothermic, the reactor stage 13 is cooled using boiling boiler feed water. The resulting steam may be exported or used for an upstream process, for example a steam reformer. The reactor stage 13 forms part of a reactor arrangement which has at least the reactor stage 13 and a phase separation apparatus 15 connected downstream of the reactor stage. The phase separation apparatus comprises at least one heat exchanger for cooling and condensing the initially gaseous raw methanol to afford the product stream 14 and a separator arranged downstream of the heat exchanger for separating the liquid raw methanol phase from the gaseous phase. The heat exchanger and the separator have not been given dedicated reference numbers.

Withdrawn from the phase separation apparatus 15 is a liquid raw methanol stream 16 which contains methanol, water and optionally condensed byproducts from the methanol synthesis. The separator of the phase separation apparatus 15 is a high-pressure separator and the raw methanol stream 16 therefore has a pressure which differs from the synthesis pressure primarily on account of unavoidable pressure drops over the reactor arrangement. The pressure of the raw methanol stream 16 is sufficiently high to be utilized for process-internal generation of mechanical and/or electrical energy. To this end the raw methanol stream 16 is supplied to a liquid decompression apparatus 19 which may be for example a decompression turbine. In the liquid decompression apparatus 19 the raw methanol stream 16 is decompressed to a pressure of about 2 bar. This pressure corresponds to the pressure prevailing in a downstream thermal separation apparatus for obtaining pure methanol (not shown). The decompressed raw methanol stream 18 is initially supplied to a further separator, here a low-pressure separator 30. This carries out a further separation of gases which remain absorbed in the liquid phase of the raw methanol in the high-pressure separator. The gases desorbed in the low-pressure separator are discharged from the process as offgas stream 31. The raw methanol stream 29 further freed of gases is supplied to a subsequent distillation for thermal separation of the raw methanol into methanol, water and byproducts (not shown).

The liquid decompression apparatus 19 does mechanical work which is converted into electrical energy using a generator (not shown). The liquid decompression apparatus 19 is in operative connection with a motor 24 which is in turn in operative connection with the synthesis gas compression apparatus 12 that is driven by the motor. In this embodiment the liquid decompression apparatus is in indirect operative connection with the synthesis gas compression apparatus 12. The liquid decompression apparatus 19 may alternatively also be in direct operative connection with the synthesis compression apparatus 12, for example via a direct mechanical coupling.

The high-pressure separator of the phase separation apparatus 15 is supplied to a residual gas stream 17 as a gaseous phase of the separation which comprises as main constituents unconverted synthesis gas (hydrogen, carbon monoxide, carbon dioxide) and the inert gas component (nitrogen, methane). This residual gas stream 17 is recompressed to synthesis pressure using a residual gas compression apparatus 27, here a recycle gas compressor, and combined with the compressed synthesis gas stream. Since in the case of continuous recycling inert constituents would be concentrated in the synthesis gas stream supplied to the reactor stage 13 a purge gas stream 20 is continuously diverted from the residual gas stream 17. This purge gas stream 20 is recycled to a gas decompression apparatus 22, here a gas expander, in which it is decompressed to atmospheric pressure. The decompressed purge gas stream 21 is either sent for flaring (not shown) or, if it comprises a sufficiently high content of valuable gases, supplied to a hydrogen recovery plant (not shown). The gas decompression apparatus 19 is, as shown above for the liquid decompression apparatus 21, also in indirect or direct operative connection with the synthesis gas compression apparatus 12.

The following numerical example demonstrates on the basis of simulation data the advantage of the process according to the invention for a configuration as described for FIG. 1.

A pilot plant produces methanol on the basis of a nitrogen-rich synthesis gas stream which contains 12.1% by volume of carbon dioxide, 12.9% by volume of carbon monoxide, 49.9% by volume of hydrogen and 25.1% by volume of nitrogen. The synthesis gas stream is supplied to the reactor at a mass flow of 8.5 kg/h and compressed using the synthesis gas compressor, from initially 4 bar to a synthesis pressure of 90 bar. A raw methanol stream is obtained at a mass flow of 3.3 kg/h and decompressed from about 90 bar to 2.1 bar via a decompression turbine. At this pressure the decompressed raw methanol stream may be directly supplied to the subsequent distillation. Utilizing the work done by the decompression turbine achieves a saving of 0.011 kW of compressor power which corresponds to a proportion of 0.6% of the total compressor power.

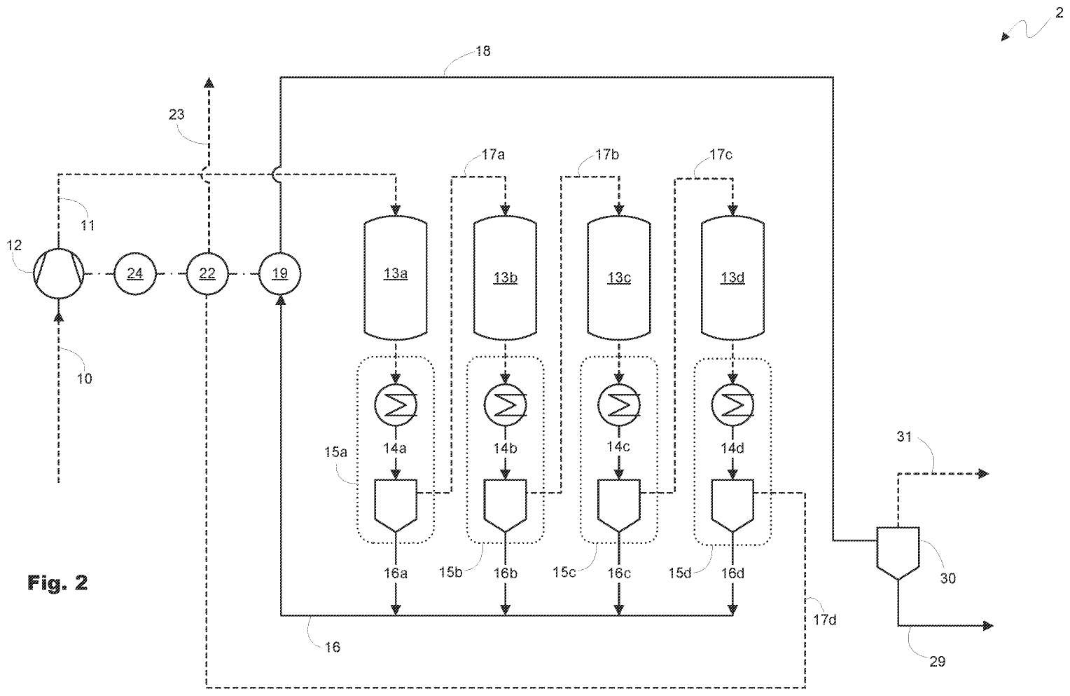

FIG. 2 shows a process 2 according to a second example of the invention in which the synthesis of the methanol is carried out in a four-stage reactor arrangement.

The reactor arrangement according to FIG. 2 comprises four serially arranged reactor stages 13a to 13d, wherein each reactor stage has a downstream phase separation apparatus assigned to it, wherein the phase separation apparatuses are correspondingly labelled 15a to 15d.

In the phase separation apparatus 15a the product substream 14a produced in reactor stage 13a is separated into a raw methanol substream 16a as a liquid phase and a residual gas substream 17a as a gaseous phase similarly to the process of FIG. 1 using a heat exchanger and a high-pressure separator. The raw methanol substream 16a is combined with further raw methanol substreams 16b, 16c and 16d, thus resulting in the raw methanol stream 16 (or overall raw methanol stream). The residual gas substream 17a withdrawn from the separator is supplied to the subsequent reactor stage 13b for further reaction of the remaining synthesis gas of the residual gas substream 17a in the second reactor stage 13b. The phase separation apparatus 15b assigned to the reactor stage 13b and arranged downstream thereof in turn produces a raw methanol substream 16b and a residual gas substream 17b. Analogous processes are carried out in the subsequent reactor stages 13c and 13d and in phase separation apparatuses 15c and 15d. Serial arrangement of the reactor stages 13a to 13d and (intermediate) condensation of the raw methanol in the phase separation apparatuses 15a to 15d makes it possible to achieve a complete or at least virtually complete carbon conversion over the entire reactor arrangement upon appropriate configuration and composition of the synthesis gas. The residual gas substream 17d withdrawn from the fourth phase separation apparatus 15d therefore only contains small residual amounts of carbon oxides and hydrogen and thus consists especially of the inert gas component.

The raw methanol stream 16 is supplied to the liquid decompression apparatus 19 for process-internal generation of mechanical or electrical energy. In terms of the configuration and the operative connections of the liquid decompression apparatus 19, the gas decompression apparatus 22, the motor 24 and the synthesis gas compression apparatus 12, the foregoing as described for FIG. 1 applies.

In contrast to process 1 according to FIG. 1, in process 2 of FIG. 2 it is not the purge stream but rather the entire residual gas stream, here residual gas substream 17d, that is supplied to the gas decompression apparatus 22. There results a decompressed residual gas substream 23 which is sent for flaring for example (not shown).

The following numerical example demonstrates on the basis of simulation data the advantage of the process according to the invention for a configuration as described for FIG. 2.

On an industrial scale, ethanol is produced on the basis of a nitrogen-rich synthesis gas stream which comprises exclusively carbon dioxide as the carbon oxide component. Since the synthesis gas stream contains no carbon monoxide, one reactor stage would be expected to result in a low equilibrium conversion to the product, which is why a four-stage methanol synthesis with intermediate condensation of the product is selected for this synthesis gas composition. The synthesis gas stream has a mass flow of 15240.1 kg/h in terms of carbon dioxide, a mass flow of 2095.5 kg/h in terms of hydrogen and a mass flow of 294.1 kg/h in terms of nitrogen. The synthesis gas stream is compressed from 10 bar to a pressure of 81 bar using the synthesis gas compressor.

This affords a raw methanol stream having a mass flow of 11258 kg/h in terms of the methanol proportion and a mass flow of 6111 kg/h in terms of the water proportion. The raw methanol stream moreover comprises a proportion of 328 kg/h of dissolved carbon dioxide which was not removed in the high-pressure separators of phase separation apparatuses 15a to 15d.

The residual gas stream withdrawn from the last of the serially arranged phase separation apparatuses comprises nitrogen with a mass flow of 643 kg/h, carbon dioxide with a mass flow of 573 kg/h, methanol with a mass flow of 78 kg/h, carbon monoxide with a mass flow of 23 kg/h, hydrogen with a mass flow of 85 kg/h and water with a mass flow of 3 kg/h. The main components of the residual gas stream are thus nitrogen as the inert gas component and carbon dioxide that is unconverted due to its low reactivity.

The raw methanol stream is decompressed from 81 bar to 2.1 bar in a decompression turbine in order to be supplied to the subsequent distillation. The residual gas substream is decompressed from 81 bar to 1 bar in an expander.

The synthesis gas compressor has an electrical energy demand of 7704 KW. The process-internally utilized energy of the decompression turbine makes it possible to reduce this demand by 116.3 KW which corresponds to an energy saving of 1.5%. Especially in the case of an industrial process these are significant savings which in the long term overcompensate for the additional apparatus costs. It is simultaneously clear from the above example that the achieved energy-saving is proportional to the scale of the process (cf. pilot scale and industrial scale).

The demand for electrical energy for the synthesis gas compressor may simultaneously be reduced by 53.4 KW on account of the process-internally utilized energy of the expander which corresponds to a further energy-saving of 0.7%. This results in an overall saving of electrical energy of 2.2%.

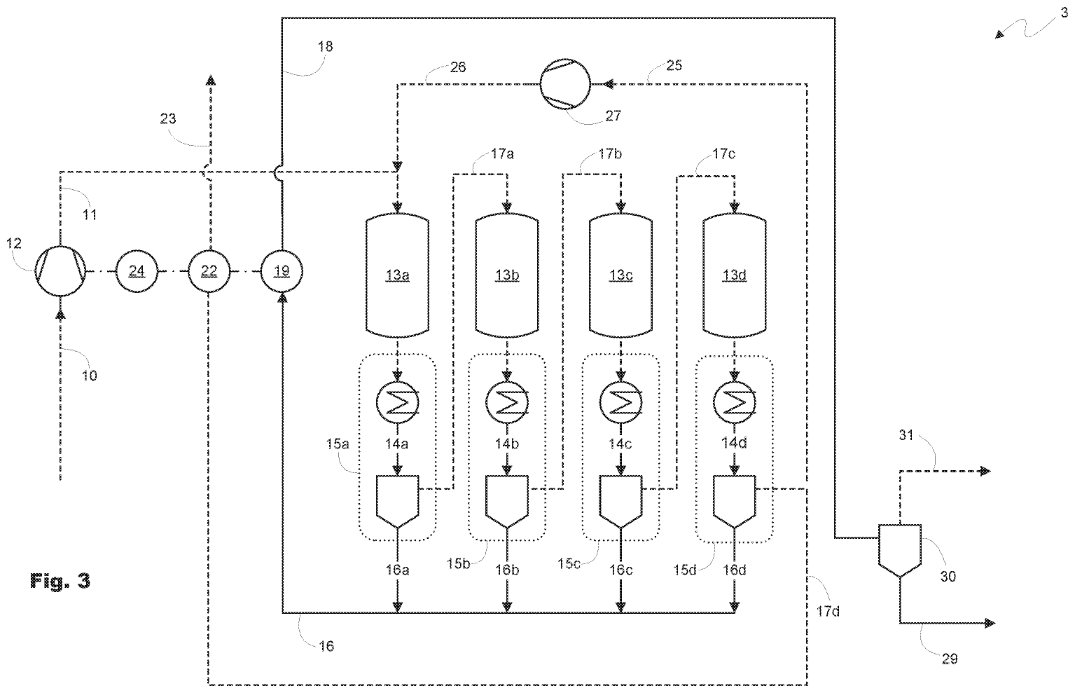

FIG. 3 shows a process 3 according to a third example of the invention in which the synthesis of the methanol is carried out in a four-stage reactor arrangement similarly to process 2.

As shown in the preceding numerical example the residual gas substream 17d may still contain valuable gases, i.e. gases convertible into methanol such as hydrogen, carbon monoxide and carbon dioxide. Depending on the proportion of these valuable gases it may be advantageous for a portion of the residual gas substream 17d to be diverted and recycled in particular to the first reactor stage of the four-stage reactor arrangement using a residual gas compression apparatus. This is realized in process 3 according to FIG. 3. Here, a substream 25 of the residual gas substream 17d is recompressed to synthesis pressure in a residual gas compression apparatus 27, here a recycle gas compressor, and as compressed substream 26 of the residual gas substream 17d combined with the previously compressed synthesis gas stream 11.

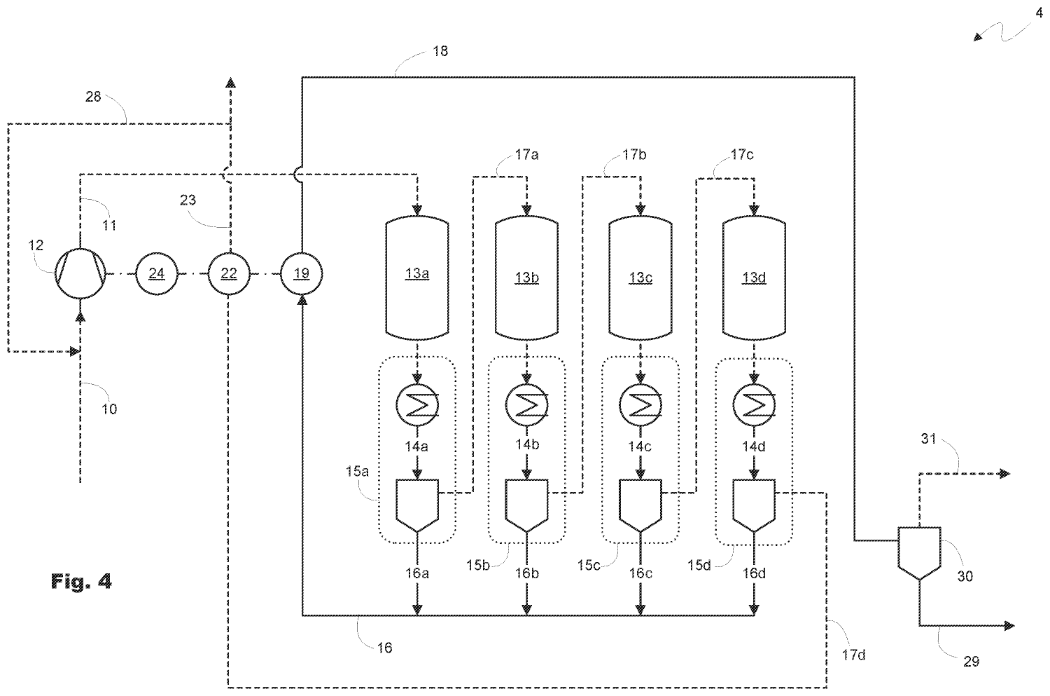

FIG. 4 shows a process 4 according to a fourth example of the invention in which the synthesis of the methanol is carried out in a four-stage reactor arrangement similarly to process 2.

The process mode according to FIG. 4 represents an alternative to process 3 in terms of the utilization of valuable gases present in the residual gas substream 17d. According to process 4 a portion of the previously decompressed residual gas substream 23 is diverted as substream 28 and combined with the still uncompressed synthesis gas stream 10. Compression is subsequently carried out in the synthesis gas compression apparatus 12. Compared to the configuration according to FIG. 3 the configuration according to FIG. 4 has the advantage that no residual gas compression apparatus 27 is required. The disadvantage of this is that the recompression of substream 28 requires additional input of electrical energy into the synthesis gas compression apparatus 12.

### LIST OF REFERENCE SYMBOLS

1, 2, 3, 4 Process according to the invention
10 Synthesis gas stream
11 Compressed synthesis gas stream
12 Synthesis gas compression apparatus (synthesis gas compressor)
13 Reactor stage
13a, 13b, 13c, 13d Reactor stage
14 Product stream
14a, 14b, 14c, 14d Product substream
15 Phase separation apparatus
15a, 15b, 15c, 15d Phase separation apparatus
16 Raw methanol stream
16a, 16b, 16c, 16d
17 Raw methanol substream
17a, 17b, 17c, 17d Residual gas stream
18 Decompressed raw methanol stream
19 Liquid decompression apparatus (decompression turbine)
20 Purge gas stream
21 Decompressed purge gas stream
22 Gas decompression apparatus (expander)
23 Decompressed residual gas substream
24 Motor
25 Substream of residual gas substream 17d
26 Compressed substream of residual gas substream 17d
27 Residual gas compression apparatus (recycle gas compressor)
28 Substream of decompressed residual gas substream 23
29 Raw methanol stream
30 Low pressure separator
31 Offgas stream

What is claimed is:

1. A process for producing methanol, comprising:
a) providing a synthesis gas stream comprising at least one carbon oxide, hydrogen and an inert gas component;
b) compressing the synthesis gas stream to synthesis pressure in a synthesis gas compression apparatus;
c) reacting the compressed synthesis gas stream over a methanol synthesis catalyst thereby producing a product stream, wherein the product stream comprises at least methanol, water, unconverted synthesis gas and the inert gas component, wherein the reacting of the compressed synthesis gas stream is performed in a reactor arrangement, wherein the reactor arrangement comprises at least one reactor stage and a phase separation apparatus;
d) separating the product stream in the phase separation apparatus into a liquid raw methanol stream, comprising at least methanol and water, and a residual gas stream comprising at least unreacted synthesis gas and the inert gas component;
e) decompressing at least a portion of the raw methanol stream in a liquid decompression apparatus to a decompression pressure which is lower than the synthesis pressure, wherein the liquid decompression apparatus does mechanical work; and
f) transmitting at least a portion of the mechanical work done by the liquid decompression apparatus to the synthesis gas compression apparatus to drive the synthesis gas compression apparatus or
converting at least a portion of the mechanical work done by the liquid decompression apparatus into electrical energy and utilizing the electrical energy to drive the synthesis gas compression apparatus.

2. The process according to claim 1, wherein the reactor arrangement comprises a plurality n of serially arranged reactor stages and a plurality p of phase separation apparatuses, wherein each of the reactor stages has a phase separation apparatus assigned to it, wherein the phase separation apparatus is in each case arranged downstream of the assigned reaction stage and in each of the phase separation apparatuses a liquid raw methanol substream and a residual gas substream are generated and wherein the residual gas substream produced in a phase separation apparatus is at least partially introduced into the respective subsequent reactor stage and wherein the residual gas substream produced in the last phase separation apparatus is discharged from the reactor arrangement.

3. The process according to claim 2, comprising the process steps of
g) decompressing at least a portion of the residual gas substream discharged from the reactor arrangement in a gas decompression apparatus to a decompression pressure lower than the synthesis pressure, wherein the gas decompression apparatus does mechanical work;
h) transmitting at least a portion of the mechanical work done by the gas decompression apparatus to the synthesis gas compression apparatus to drive the synthesis gas compression apparatus or
converting at least a portion of the mechanical work done by the gas decompression apparatus into electrical energy and utilizing the electrical energy to drive the synthesis gas compression apparatus.

4. The process according to claim 3, wherein the entire residual gas stream discharged from the reactor arrangement is decompressed in the gas decompression apparatus.

5. The process according to claim 3, wherein a proportion of the residual gas substream discharged from the reactor arrangement is decompressed in the gas decompression apparatus and a further proportion of the residual gas substream discharged from the reactor arrangement is compressed to synthesis pressure in a residual gas compression apparatus and the compressed residual gas substream is recycled at least to one of the plurality of serially arranged reactor stages.

6. The process according to claim 3, wherein at least a portion of the residual gas substream decompressed to decompression pressure is recompressed to synthesis pressure in the synthesis gas compression apparatus and the compressed residual gas substream is supplied to the reactor arrangement.

7. The process according to claim 1, wherein the proportion of the inert gas component in the synthesis gas stream is at least 1% by volume.

8. The process according to claim 1, wherein the inert gas component comprises nitrogen and/or methane.

9. The process according to claim 1, wherein the synthesis pressure is at least 70 bar.

10. The process according to claim 1, wherein the raw methanol stream in the liquid decompression apparatus is decompressed to a pressure of 1 bar to 3 bar.

11. The process according to claim 1, wherein the raw methanol stream is supplied to a downstream thermal separation apparatus for separating the raw methanol into methanol and water, wherein the thermal separation apparatus is operated at a predetermined pressure and the raw methanol stream is decompressed in the liquid decompression apparatus to a pressure corresponding to the predetermined pressure in the thermal separation apparatus.

12. The process according to claim 2, wherein the residual gas substream is decompressed to a pressure of 1 bar to 3 bar in the gas decompression apparatus.

13. The process according to claim 2, wherein for the plurality n of serially arranged reactor stages and the plurality p of phase separation apparatuses
n=3 to 6, and
p=3 to 6, and wherein
n=p.
